(19) United States
(12) Patent Application Publication
(10) Pub. No.: US 2010/0047258 A1
(43) Pub. Date: Feb. 25, 2010

(54) Drug Carriers, Their Synthesis, and Methods of Use Thereof

(76) Inventors: Dong Wang, Omaha, NE (US); Richard A. Reinhardt, Roca, NE (US); Xin-Ming Liu, Omaha, NE (US)

Correspondence Address: DANN, DORFMAN, HERRELL & SKILLMAN, 1601 MARKET STREET, SUITE 2400, PHILADELPHIA, PA, 19103-2307, US

(21) Appl. No.: 12/374,387

(22) PCT Filed: Aug. 2, 2007
(86) PCT No.: PCT/US07/75073

§ 371 Date: Sep. 23, 2009

Related U.S. Application Data
(60) Provisional application No. 60/834,924, filed on Aug. 2, 2006.
(60) Provisional application No. 60/854,848, filed on Oct. 27, 2006.
(60) Provisional application No. 60/896,604, filed on Mar. 23, 2007.

(51) Int. Cl. A61K 39/395; C07K 16/00; C08B 37/16; A61K 31/724; A61P 37/06

(52) U.S. Cl. 424/178.1; 530/391.1; 536/103; 514/58

(57) ABSTRACT

Drug carriers, methods of synthesizing, and methods of use thereof are provided.

This application claims priority under 35 U.S.C. §119(e) to U.S. Provisional Patent Application No. 60/834,924, filed on Aug. 2, 2006; U.S. Provisional Patent Application No. 60/854,848, filed on Oct. 27, 2006; and U.S. Provisional Patent Application No. 60/896,604, filed on Mar. 23, 2007. The foregoing applications are incorporated by reference herein.

### FIELD OF THE INVENTION

The present invention relates to drug carriers and methods of use thereof. More specifically, the instant invention relates to hard tissue targeting-cyclodextrins and multifunctional poly(ethylene glycol) (PEG).

### BACKGROUND OF THE INVENTION

Several publications and patent documents are cited throughout the specification in order to describe the state of the art to which this invention pertains. Each of these citations is incorporated herein by reference as though set forth in full.

Bone is a highly specified form of connective tissue, which provides an internal support system in all vertebrates. To maintain its normal function, bone is continuously being resorbed and rebuilt throughout the skeleton. In healthy individuals, bone resorption and formation are well balanced with the bone mass maintained in a steady state. Disturbances of this balance are characteristic of a number of bone diseases including osteoporosis, Paget's disease, osteopetrosis, bone cancer, etc. (Odgren et al. (2000) Science 289:1508-1514). Currently, 44 million Americans, or 55% of the people 50 years of age and older, are in danger of having osteoporosis; 10 million individuals probably already have the disease (Burckhardt et al. (1991) Am. J. Med., 90:107-110; America's Bone Health: The State of Osteoporosis and Low Bone Mass in Our Nation; National Osteoporosis Foundation Washington, D.C., 2002; pp 1-16). Similarly, arthritis, such as rheumatoid arthritis and osteoarthritis, which is always accompanied by skeletal complications, also affect tens of millions of American lives (O'Dell, J. R. (2004) N. Engl. J. Med., 350:2591-2602; Firestein, G. S. Etiology and Pathogenesis of Rheumatoid Arthritis. In Kelley's Textbook of Rheumatology, 7th ed.; Harris, E. D., et al., Eds.; Elsevier Saunders: Philadelphia, 2005; p. 996; Wieland et al. (2005) Nat. Rev. Drug Discovery 4:331-344).

Rheumatoid arthritis (RA) is a chronic, systemic, inflammatory disease, which involves the destruction of joints. It is often considered to be an autoimmune disorder, though the exact cause of the disease is unknown. The primary target of the disease is synovial tissue. The inflamed synovium tissue (including synovial fibroblasts and osteoclasts) invades and damages articular bone and cartilage, leading to significant pain and loss of movement. Currently, RA affects approximately 0.8 percent of adults worldwide, has an earlier onset and is more common in women than men, frequently beginning in the childbearing years. When the disease is unchecked, it often leads to substantial disability and premature death (O'Dell, J. R. (2004) N. Engl. J. Med., 350:2591-2602; Firestein, G. S. (2005) Etiology and Pathogenesis of Rheumatoid Arthritis. In Kelley's Textbook of Rheumatology, 7th Ed. Elsevier Saunders, Philadelphia, 996; McDuffie, F. C. (1985) Am. J. Med., 78:1-5).

### SUMMARY OF THE INVENTION

In accordance with the instant invention, compounds are provided which target biominerals such as bone and teeth. In a particular embodiment, the compounds are of the general formula T-X-CD, wherein X is a linker domain, T is bone targeting moiety, and CD is a cyclodextrin. In a particular embodiment, the bone targeting moiety is alendronate.

In accordance with another aspect of the instant invention, compositions are provided which comprise the bone targeting cyclodextrin compound of the instant invention and at least one pharmaceutically acceptable carrier. The compositions may further comprise at least one therapeutic agent which may optionally be contained within the cavity of the cyclodextrin. In a particular embodiment, the therapeutic agent is a bone related therapeutic agent.

In yet another aspect of the invention, methods of preventing or treating bone disorders and bone disorder-related conditions or complications in a subject in need thereof are provided. The methods comprise administering to the patient the pharmaceutical composition of the instant invention. The compositions may be administered systemically or locally.

In accordance with another embodiment of the instant invention, multifunctional PEGs are provided. The multifunctional PEG may comprise a copolymer of PEG blocks linked by “click” polymerization reactions. In a particular embodiment, the drug carrier is formula I.

In accordance with another aspect of the instant invention, compositions are provided which comprise the multifunctional PEG and at least one pharmaceutically acceptable carrier. The compositions may further comprise at least one therapeutic agent.

### BRIEF DESCRIPTION OF THE DRAWING

FIG. 1 provides an exemplary T-X-CD wherein cyclodextrin is connected to alendronate (the bone targeting moiety) via a linker moiety.

FIG. 2 provides a schematic scheme for conjugating alendronate to cyclodextrin.

FIGS. 3A-3E provide graphs of the infiltrate size (mm²), percent lymphocytes (lateral), new bone area (mm²±SEM), new bone width (mm±SEM), and percent of osteoblast (lateral), respectively, obtained from the analyses of the images of hematoxylin and eosin stained, decalcified sections of the mandible of rats treated with different formulations. 1 is prostaglandin E₁ (PGE₁)/alendronate (ALN)-cyclodextrin (CD), 2 is PGE₁/hydroxypropyl (HP)-β-CD, 3 is PGE₁/ALN-CD plus BioOss®, 4 is PGE₁/HP-β-CD plus BioOss®, 5 is ALN-CD, and 6 is HP-β-CD. **p<0.01, ***p<0.001.

FIGS. 4A-4G provide images of hematoxylin and eosin stained, decalcified sections of the mandible of rats treated with PGE₁/ALN-CD (FIG. 4A), PGE₁/HP-β-CD (FIG. 4B), PGE₁/ALN-CD plus BioOss® (FIG. 4C), PGE₁/HP-β-CD plus BioOss® (FIG. 4D), ALN-CD (FIG. 4E), and HP-β-CD (FIG. 4F). FIG. 4G is a 200× magnification of FIG. 4A. White arrow points to the mandible, grey arrow points to new bone, and black arrow points to the BioOss® particles.

FIG. 5 is a schematic of the synthesis of linear multifunctional PEG via Cu(I)-catalyzed Huisgen 1,3-dipolar cycloaddition.

FIG. 6 provides graphs of the ¹H NMR spectra (D₂O) of acetylene terminated PEG 2000 (FIG. 6A) and linear multifunctional PEG obtained via “click” reaction (FIG. 6B).

FIG. 7 is a graph of the size-exclusion chromatography (SEC) analysis of “click” polymerization product. Superose 6 column (HR 10/30) was used with PBS (pH=7.3) as eluent. Polyethylene oxide (PEO) calibration sample (MW=66 kDa) was used as a reference. Arrow represents a small amount of unreacted acetylene-terminated PEG 2000.

### DETAILED DESCRIPTION OF THE INVENTION

### I. Bone-Targeting Drug Carrier

In one embodiment, the instant invention pertains to hard tissue (e.g., bone and teeth) targeting compounds and methods of use thereof. Preferably, the targeting compounds are of the formula: T-X-CD, wherein X is a linker domain, T is a bone targeting moiety or moieties, and CD is a cyclodextrin.

While hydroxypropyl(HP)-β-CD is exemplified hereinbelow, other cyclodextrins may be used in the compounds of the instant invention including, without limitation, α-CD, β-CD, γ-CD, μ-CD, and derivatives thereof such as dimethyl-β-CD, carboxymethyl-ethyl-β-CD, sulfobutyl-ethyl-β-CD, and those described in U.S. Pat. Nos. 4,727,064 and 5,376,645. The compounds of the instant invention comprise at least one type of cyclodextrin. In a preferred embodiment, each cyclodextrin is linked to at least one bone targeting moiety. The cyclodextrin hydrophobic cavity may be free or available (i.e., the cyclodextrin cavity is not loaded with a therapeutic compound or drug) or may be loaded or complexed with a therapeutic compound or drug.

The cyclodextrin of the compounds of the instant invention may also be cyclodextrin polymers (i.e., cyclodextrins joined together by covalent bonds). The cyclodextrin polymers may be linear, branched, or dendritic polymers. The cyclodextrin polymers may comprise about 2 to about 200 cyclodextrin units.

The linker domain X is a chemical moiety comprising a covalent bond or a chain of atoms that covalently attaches the bone targeting moiety to the cyclodextrin. In a particular embodiment, the linker may contain from 0 (i.e., a bond) to about 500 atoms, about 1 to about 100 atoms, or about 1 to about 50 atoms. The linker can be linked to any synthetically feasible position of cyclodextrin. In a preferred embodiment the linker is attached at a position which avoids blocking the drug binding cavity of cyclodextrin (e.g., on the outside of the cyclodextrin ring). Exemplary linkers may comprise at least one optionally substituted; saturated or unsaturated; linear, branched or cyclic alkyl, alkenyl, or aryl group. The linker may also be a polypeptide (e.g., from about 1 to about 20 amino acids). The linker may be biodegradable under physiological environments or conditions. The linker may also be non-degradable and may be a covalent bond or any other chemical structure which cannot be cleaved under physiological environments or conditions.

Bone targeting moieties (T) are those compounds which preferentially accumulate in hard tissue or bone rather than any other organ or tissue in vivo. Bone targeting moieties of the instant invention include, without limitation, bisphosphonates (e.g., alendronate), tetracycline, sialic acid, malonic acid, N,N-dicarboxymethylamine, 4-aminosalicyclic acid, 4-aminosalicyclic acid, bone targeting antibodies or fragments thereof, and peptides (e.g., peptides comprising about 2 to about 100 D-glutamic acid residues, L-glutamic acid residues, D-aspartic acid residues, and/or L-aspartic acid residues). In a preferred embodiment, the bone targeting moiety is alendronate, thereby resulting in a compound of the formula ALN-X-CD, wherein X is a linker domain.

Compositions comprising the bone targeting cyclodextrin are also encompassed by the instant invention. The compositions comprise at least one pharmaceutically acceptable carrier. The composition may also further comprise at least one antibiotic, anti-inflammatory drug, anesthetic, and/or “bone related therapeutic agent.” A “bone related therapeutic agent” refers to an agent suitable for administration to a patient that induces a desired biological or pharmacological effect such as, without limitation, 1) increasing bone growth, 2) preventing an undesired biological effect such as an infection, 3) alleviating a condition (e.g., pain or inflammation) caused by a disease associated with bone, and/or 4) alleviating, reducing, or eliminating a disease from bone. Preferably, the bone related therapeutic agent possesses a bone anabolic effect and/or bone stabilizing effect. Bone related therapeutic agents include, without limitation, cathepsin K inhibitor, metalloproteinase inhibitor, prostaglandin E receptor agonist, prostaglandin E1 or E2 and analogs thereof, parathyroid hormone and fragments thereof, glucocorticoids (e.g., dexamethasone) and derivatives thereof, and statins (e.g., simvastatin). The bone related therapeutic agent may be covalently linked (optionally via a linker domain) to the bone targeting cyclodextrin (T-X-CD) of the instant invention, particularly to the cyclodextrin molecule. In a preferred embodiment, the bone related therapeutic agent is bound to the bone targeting cyclodextrin by other physical interactions such as to the hydrophobic cavity of cyclodextrin via, for example, van der Waals forces.

The pharmaceutical compositions of the present invention can be administered by any suitable route, for example, by injection, oral, pulmonary, or other modes of administration. The compositions of the instant invention may be administered locally or systemically (e.g., for treating osteoporosis). In a preferred embodiment, the composition is injected directly to the desired site.

The pharmaceutical compositions of the present invention may be delivered in a controlled release system, such as via an implantable osmotic pump or other mode of administration. In another embodiment, polymeric materials may be employed to control release (see Medical Applications of Controlled Release, Langer and Wise (eds.), CRC Press: Boca Raton, Fla. (1974); Controlled Drug Bioavailability, Drug Product Design and Performance, Smolen and Ball (eds.), Wiley: New York (1984); Ranger and Peppas, J. Macromol. Sci. Rev. Macromol. Chem. (1983) 23:61; see also Levy et al., Science (1985) 228:190; During et al., Ann. Neurol. (1989) 25:351; Howard et al., J. Neurosurg. (1989) 71:105). The controlled release system may be placed in proximity of the target area of the subject. Other potential controlled release systems are discussed in the review by Langer (Science (1990) 249:1527 1533).

Compositions of the instant invention may also be administered as part of a medical device. As used herein, the term “medical device” includes devices and materials that are permanently implanted and those that are temporarily or transiently present in the patient. The compositions of the invention can be released from the medical devices or coated on the medical devices. Medical devices include, without limitation, stents, plates, fracture implants, gels, polymers (e.g., sustained release polymers or gels), and release devices.

The compositions of the invention may also be coated on or administered with grafts and implants such as, without limitation, dura mater grafts, cartilage grafts, cartilage implants, bone grafts, bone implants, and bone marrow grafts.

The present invention is also directed to methods of preventing or treating bone disorders and bone disorder-related conditions or complications in a subject that is in need of such prevention or treatment, comprising administering to the patient a composition of the instant invention. Bone disorders may be associated with bone loss and include, without limitation, osteoporosis, osteopenia, bone fractures, bone breaks, Paget's disease (osteitis deformans), bone degradation, bone weakening, skeletal distortion, low bone mineral density, scoliosis, osteomalacia, osteomyelitis, osteogenesis imperfecta, osteopetrosis, enchondromatosis, osteochondromatosis, achondroplasia, alveolar bone defects, spine vertebra compression, bone loss after spinal cord injury, avascular necrosis, fibrous dysplasia, periodontal disease, hyperparathyroidism (osteitis fibrosa cystica), hypophosphatasia, fibrodysplasia ossificans progressive, and pain and inflammation of the bone. Bone related therapeutic agents can be administered in the same composition as the bone targeting-cyclodextrin compound of the instant invention or may be administered in a separate composition either concurrently or at a different time.

### II. Multifunctional PEG

In accordance with another aspect of the instant invention, novel multifunctional poly(ethylene glycol) (PEG) copolymers and methods of synthesizing the same are provided. PEG is a water-soluble, highly biocompatible synthetic polymer that has been widely used in drug delivery and bioconjugation. It is known to be nonimmunogenic and has superior biocompatibility (Chapman et al. (2002) Adv. Drug Deliv. Rev., 54:531-545; Greenwald et al. (2003) Adv. Drug Deliv. Rev., 55:217-250). Several PEG conjugated (PEGylated) therapeutic agents have been approved by FDA for various clinical applications (Duncan, R. (2003) Nat. Rev. Drug Discov., 2, 347-360; Veronese et al. (2005) Drug Discov. Today, 10, 1451-8; Shen et al. (2006) Curr. Opin. Mol. Ther., 8, 240-248). However, only chain termini-functionalized PEG has been used so far because of the difficulties associated with synthesizing linear multifunctional PEG. Improvement of its limited functionality (two chain termini) would significantly expand its current applications. The present invention offers a very simple way of synthesizing multifunctional PEG. The synthesis and adjustment of the functionality of the PEG conjugates of the instant invention can be easily accomplished, which makes personalized macromolecular therapy a possibility. Additionally, biodegradation structures (e.g., an ester bond) can be introduced into the polymer main chain, thereby making the high molecular weight PEG biodegradable. The degraded PEG can then be eliminated from the system, thereby greatly enhancing the biocompatibility of PEG. The multifunctional PEG also has a well-defined structure as each functional group can be divided by a short but well-defined PEG chain.

Hereinbelow, a simple and yet highly efficient strategy in the synthesis of linear multifunctional PEGs with “click” chemistry is provided. Short acetylene-terminated PEG was linked by 2,2-bis(azidomethyl)propane-1,3-diol using Cu(I)-catalyzed Huisgen 1,3-dipolar cycloaddition in water at room temperature. High molecular weight PEGs with pendent hydroxyl groups were obtained and characterized by ¹H NMR and size-exclusion chromatography (SEC). This simple “click” polymerization approach provides a powerful tool for the development of novel polymers and functional polymer conjugates for biomedical applications.

Click chemistry refers to a set of covalent bond-forming reactions between two functional groups with high yields that can be performed under extremely mild conditions (Kolb et al. (2001) Angew. Chem. Int. Ed., 40:2004-2021; Lewis et al. (2002) Angew. Chem. Int. Ed., 41:1053-1057). Click reactions are generally a reaction between a carbon atom and a heteroatom that is irreversible, highly energetically favored, goes largely to completion, and occurs between two groups that are generally unreactive except with respect to each other. Click chemistry techniques are described, for example, in the following references: U.S. Pat. No. 7,208,243; U.S. Patent Application Publication Nos.: 2006/0154129, 2006/0269942, 2005/0222427, and 2006/0263293; Kolb et al. (2001) Angew. Chem. Intl. Ed., 40:2004-2021; Kolb et al. (2003) Drug Disc. Tod., 8:1128-1137; Rostovtsev et al. (2002) Angew. Chem. Intl. Ed., 41:2596-2599; Tomoe et al. (2002) J. Org. Chem., 67:3057-3064; Wang et al. (2003) J. Amer. Chem. Soc., 125:3192-3193; Lee et al. (2003) J. Amer. Chem. Soc., 125:9588-9589; Lewis et al. (2002) Angew. Chem. Int. Ed., 41:1053-1057; Manetsch et al. (2004) J. Amer. Chem. Soc., 126:12809-12818; and Mocharla et al. (2005) Angew. Chem. Int. Ed., 44:116-120. Any click chemistry functional groups can be utilized in the instant invention. In a particular embodiment, cycloaddition reactions are used, such as the Huisgen 1,3-dipolar cycloaddition of azides and alkynes in the presence of Cu(I) salts thereby forming 1,4-disubstituted 1,2,3-triazoles (see, e.g. Padwa, A., ed., Huisgen 1,3-Dipolar Cycloaddition Chemistry (Vol. 1), Wiley, pp. 1-176; Jorgensen (2000) Angew. Chem. Int. Ed. Engl., 39:3558-3588; Tietze et al. (1997) Top. Curr. Chem., 189:1-120). Alternatively, in the presence of Ru(II) salts, terminal alkynes or alkynyls and azides undergo 1,3-dipolar cycloaddition to form 1,5-disubstituted 1,2,3-triazoles (Fokin et al. (2005) Organ. Lett., 127:15998-15999; Krasinski et al. (2004) Organ. Lett., 1237-1240).

The Cu(I)-catalyzed variant of the Huisgen 1,3-dipolar cycloaddition of azides and alkynes to form 1,2,3-triazoles has emerged as the most reported “click” reaction. It is characterized by high reaction yields, mild reaction conditions, tolerance of oxygen and water, simple workup, good functional group compatibility and strong reliability (Rostovtsev et al. (2002) Angew. Chem. Int. Ed., 41:2596-2599; Bock et al. (2006) Eur. J. Org. Chem., 51-68). When 2,2-bis(azidomethyl)propane-1,3-diol was used as a difunctional azide reactant, an extremely high reaction rate was observed potentially due to a self-catalyzing mechanism (Rodionov et al. (2005) Angew. Chem. Int. Ed., 44:2210-2215). Practically, it is easy to introduce azides and acetylenes into organic compounds and these structures are stable under other reaction conditions. These unique characteristics have made the Cu(I)-catalyzed Huisgen 1,3-dipolar cycloaddition a powerful linking reaction in drug discovery (Kolb et al. (2003) Drug Discov. Today., 8:1128-1137; Manetsch et al. (2004) J. Am. Chem. Soc., 126:12809-12818; Mocharla et al. (2005) Angew. Chem., Int. Ed., 44:116-120), polymer synthesis (Parrish et al. (2005) J. Am. Chem. Soc., 127:7404-7410; Malkoch et al. (2005) J. Am. Chem. Soc., 127:14942-14949; Ladmiral et al. (2006) J. Am. Chem. Soc., 128:4823-4830; Wu et al. (2004) Angew. Chem. Int. Ed., 43:3928-3932), nanoparticle (Joralemon et al. (2005) J. Am. Chem. Soc., 127:16892-16899), and biomacromolecule functionalization (Wang et al. (2003) J. Am. Chem. Soc., 125:3192-3193; Beatty et al. (2005) J. Am. Chem. Soc., 127:14150-14151).

The PEG multifunctional copolymers of the instant invention consisting of modified PEG blocks linked by click chemistry, such as by 2,2-bis(azidomethyl)-propane-1,3-diol, provide a water-soluble, polymeric drug delivery system. The multifunctional PEG is a general drug delivery platform that can be used as drug carrier for macromolecular therapy. The multifunctional PEG may be generated by performing a click reaction between a modified PEG comprising a first click reaction functional group (e.g., an alkyne) at its termini with a compound comprising at least one (preferably at least two) second click reaction functional group (e.g., an azide) and, optionally, at least one other functional group (i.e., a group which reacts readily with another molecule to form a bond) which is not involved in the click reaction but rather allows for the addition of other compounds such as a therapeutic agent to the resultant multifunctional PEG. Alternatively, the compound may already be conjugated to the other compounds or therapeutic agent prior to the click reaction. For example, 2,2-bis(azidomethyl)-propane-1,3-diol and its analogs can be linked to any compound of interest. Therefore, therapeutic agents, medical imaging contrast agents, biochemical markers, targeting moieties, fluorescent markers, and other compounds could be linked to 2,2-bis(azidomethyl)-propanel-1,3-diol and introduced onto the high molecular weight PEG with a desired ratio.

A general formula of a multifunctional PEG of the instant invention is (formula I):

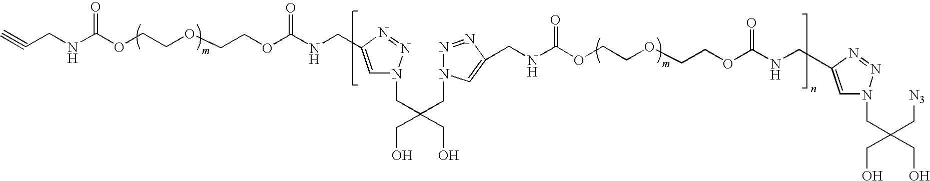

wherein m is 2 to 4000 or 2 to 1000 and n is 2 to 1000.

Clinically, the multifunctional PEG can be used as a drug delivery system to treat any disease or disorder. In a particular embodiment, the multifunctional PEG can be used for the improved treatment of solid tumor, rheumatoid arthritis and other pathological conditions with leaky vasculature. Similarly, when contrast agents or fluorescent markers are introduced into the multifunctional PEG, it can be used as a diagnostic or research tool, such as a macromolecular blood pool imaging contrast agent. Additionally, because of its very high molecular weight and viscosity, the multifunctional PEG of the instant invention may be applied directly to wound dressings, adhesive bandages, sutures, on wounds, burns, abrasions, and cuts, optionally complexed with at least one therapeutic compound drug.

The multifunctional PEG can also be used to selectively deliver anti-inflammatory compounds and immunosuppressive agents such as glucocorticoids to sites of joint inflammation in patients with inflammatory arthritis. The multifunctional copolymer may also be used for attachment of anti-rheumatoid arthritis drugs, such as dexamethasone via acetal formation. Acetal is the structure responsible for the pH-sensitive dexamethasone release.

There is no cure for rheumatoid arthritis at present. The most commonly used medications for clinical treatment and management of the disease include: nonsteroidal anti-inflammatory drugs (NSAIDs), glucocorticosteroids (GC) and disease-modifying antirheumatic drugs (DMARDs). DMARDs in combination with others are considered quite effective in controlling the disease progression (O'Dell, J. R. (2004) N. Engl. J. Med., 350:2591-2602; Smolen et al. (2003) Nat. Rev. Drug Discov., 2:473-488). While progress has been made in understanding the molecular mechanisms and identification of novel therapeutic targets for rheumatoid arthritis, the lack of arthrotropicity of most of the anti-rheumatic drugs is still a challenge. The multifunctional PEG copolymers of the instant invention provide a means for selectively delivering anti-rheumatic drugs or drug candidates to arthritic joints.

As stated hereinabove, a multifunctional PEG-based drug carrier system is provided herein where acetylene modified PEG blocks are connected, for example, by 2,2-bis(azidomethyl)-propane-1,3-diol. The copolymer may be made biodegradable by modifying PEG with, e.g., an oligopeptide, prior to capping it with acetylene. The diol from the linker is a natural structure for conjugation with carbonyl containing drugs and the formed acetal linkage is a pH-sensitive linker that has been widely used in prodrug design. The instant design also carries the advantages of simple reaction conditions and significant potential for mass production. The conjugation of drugs to this polymeric carrier is easier compared to other copolymers such as HPMA (Anderson et al. (2004) The 26th Ann. Meeting Amer. Soc. Bone Miner. Res., Seattle, Wash., October, 2004, poster presentation). Additionally, targeting moieties can also be easily introduced by modification of 2,2-bis-(azidomethyl)-propane-1,3-diol.

Compositions comprising the multifunctional PEG are also encompassed by the instant invention. The compositions comprise at least one pharmaceutically acceptable carrier. The composition may also further comprise at least one therapeutic compound, optionally linked to the multifunctional PEG. The compositions comprising the multifunctional PEG can be administered by any suitable route, for example, by injection, oral, pulmonary, or other modes of administration. The compositions of the instant invention may be administered locally or systemically (e.g., for treating osteoporosis). The compositions may also be delivered in a controlled release system, such as an implantable osmotic pump, medical device, polymeric materials, or other modes of administration. The compositions may also be coated on or administered with grafts.

### III. DEFINITIONS

The term “substantially pure” refers to a preparation comprising at least 50-60% by weight of a given material (e.g., nucleic acid, oligonucleotide, protein, etc.). More preferably, the preparation comprises at least 75% by weight, and most preferably 90-95% by weight of the given compound. Purity is measured by methods appropriate for the given compound (e.g. chromatographic methods, agarose or polyacrylamide gel electrophoresis, HPLC analysis, and the like).

The term “isolated” refers to the separation of a compound from other components present during its production. “Isolated” is not meant to exclude artificial or synthetic mixtures with other compounds or materials, or the presence of impurities that do not substantially interfere with the fundamental activity, and that may be present, for example, due to incomplete purification, or the addition of stabilizers.

“Linker”, “linker domain”, and “linkage” refer to a chemical moiety comprising a covalent bond or a chain of atoms that covalently attaches, for example, a bone targeting moiety to a cyclodextrin. In various embodiments, a linker is specified as X. The linker can be linked to any synthetically feasible position of cyclodextrin, but preferably in such a manner as to avoid blocking the drug binding cavity of cyclodextrin (i.e., on the outside of the cyclodextrin ring). Linkers are generally known in the art. Exemplary linkers may comprise at least one optionally substituted; saturated or unsaturated; linear, branched or cyclic alkyl group or an optionally substituted aryl group. The linker may also be a polypeptide (e.g., from about 1 to about 20 amino acids). The linker may be biodegradable under physiological environments or conditions. The linker may also be may be non-degradable and can be a covalent bond or any other chemical structure which cannot be cleaved under physiological environments or conditions.

As used herein, the term “bone-targeting” refers to the capability of preferentially accumulating in hard tissue rather than any other organ or tissue, after administration in vivo.

As used herein, the term “biodegradable” or “biodegradation” is defined as the conversion of materials into less complex intermediates or end products by solubilization hydrolysis under physiological conditions, or by the action of biologically formed entities which can be enzymes or other products of the organism. The term “non-degradable” refers to a chemical structure that cannot be cleaved under physiological condition, even with any external intervention. The term “degradable” refers to the ability of a chemical structure to be cleaved via physical (such as ultrasonication), chemical (such as pH of less than 4 or more than 9) or biological (enzymatic) means.

A “therapeutically effective amount” of a compound or a pharmaceutical composition refers to an amount effective to prevent, inhibit, or treat the symptoms of a particular disorder or disease. For example, “therapeutically effective amount” may refer to an amount sufficient to modulate bone loss or osteoporosis in an animal, especially a human, including, without limitation, decreasing or preventing bone loss or increasing bone mass.

“Pharmaceutically acceptable” indicates approval by a regulatory agency of the Federal or a state government or listed in the U.S. Pharmacopeia or other generally recognized pharmacopeia for use in animals, and more particularly in humans.

A “carrier” refers to, for example, a diluent, adjuvant, preservative (e.g., Thimersol, benzyl alcohol), anti-oxidant (e.g., ascorbic acid, sodium metabisulfite), solubilizer (e.g., Tween 80, Polysorbate 80), emulsifier, buffer (e.g., Tris HCl, acetate, phosphate), bulking substance (e.g., lactose, mannitol), excipient, auxiliary agent or vehicle with which an active agent of the present invention is administered. Pharmaceutically acceptable carriers can be sterile liquids, such as water and oils, including those of petroleum, animal, vegetable or synthetic origin, such as peanut oil, soybean oil, mineral oil, sesame oil and the like. Water or aqueous saline solutions and aqueous dextrose and glycerol solutions are preferably employed as carriers, particularly for injectable solutions. The compositions can be incorporated into particulate preparations of polymeric compounds such as polylactic acid, polyglycolic acid, etc., or into liposomes or micelles. Such compositions may influence the physical state, stability, rate of in vivo release, and rate of in vivo clearance of components of a pharmaceutical composition of the present invention. The pharmaceutical composition of the present invention can be prepared, for example, in liquid form, or can be in dried powder form (e.g., lyophilized). Suitable pharmaceutical carriers are described in “Remington's Pharmaceutical Sciences” by E. W. Martin (Mack Publishing Co., Easton, Pa.); Gennaro, A. R., Remington: The Science and Practice of Pharmacy, 20th Edition, (Lippincott, Williams and Wilkins), 2000; Liberman, et al., Eds., Pharmaceutical Dosage Forms, Marcel Decker, New York, N.Y., 1980; and Kibbe, et al., Eds., Handbook of Pharmaceutical Excipients (3.sup.rd Ed.), American Pharmaceutical Association, Washington, 1999.

The term “alkyl,” as employed herein, includes both straight and branched chain hydrocarbons containing about 1 to 20 carbons, preferably about 5 to 15 carbons in the normal chain. The hydrocarbon chain of the alkyl groups may be interrupted with oxygen, nitrogen, or sulfur atoms. Examples of suitable alkyl groups include methyl, ethyl, propyl, isopropyl, butyl, t-butyl, isobutyl, pentyl, hexyl, isohexyl, heptyl, 4,4 dimethylpentyl, octyl, 2,2,4 trimethylpentyl, nonyl, decyl, the various branched chain isomers thereof, and the like. Each alkyl group may optionally be substituted with 1 to 4 substituents which include, for example, halo, —OH, and alkyl.

The term “cyclic alkyl” or “cycloalkyl,” as employed herein, includes cyclic hydrocarbon groups containing 1 to 3 rings which may be fused or unfused. Cycloalkyl groups may contain a total of 3 to 20 carbons forming the ring(s), preferably 6 to 10 carbons forming the ring(s). Optionally, one of the rings may be an aromatic ring as described below for aryl. Cycloalkyl groups may contain one or more double bonds. The cycloalkyl groups may also optionally contain substituted rings that includes at least one, and preferably from 1 to about 4 sulfur, oxygen, or nitrogen heteroatom ring members. Each cycloalkyl group may be optionally substituted with 1 to about 4 substituents such as alkyl (an optionally substituted straight, branched or cyclic hydrocarbon group, optionally saturated, having from about 1-10 carbons, particularly about 1-4 carbons), halo (such as F, Cl, Br, I), haloalkyl (e.g., CCl₃ or CF₃), alkoxyl, alkylthio, hydroxy, methoxy, carboxyl, oxo, epoxy, alkyloxycarbonyl, alkylcarbonyloxy, amino, carbamoyl (e.g., NH₂C(═O)— or NHRC(═O)—, wherein R is an alkyl), urea (—NHCONH₂), alkylurea, aryl, ether, ester, thioester, nitrile, nitro, amide, carbonyl, carboxylate and thiol.

“Alkenyl” refers to an unsubstituted or substituted hydrocarbon moiety comprising one or more carbon to carbon double bonds (i.e., the alkenyl group is unsaturated) and containing from about 2 to about 20 carbon atoms or from about 5 to about 15 carbon atoms, which may be a straight, branched, or cyclic hydrocarbon group. When substituted, alkenyl groups may be substituted at any available point of attachment. Exemplary substituents may include, but are not limited to, alkyl, halo, haloalkyl, alkoxyl, alkylthio, hydroxyl, methoxy, carboxyl, oxo, epoxy, alkyloxycarbonyl, alkylcarbonyloxy, amino, carbamoyl, urea, alkylurea, and thiol. Preferably, the alkenyl group comprises alternating double and single bonds such that bonds are conjugated.

The term “aryl,” as employed herein, refers to monocyclic and bicyclic aromatic groups containing 6 to 10 carbons in the ring portion. Examples of aryl groups include, without limitation, phenyl, naphthyl, such as 1-naphthyl and 2-naphthyl, indolyl, and pyridyl, such as 3-pyridyl and 4-pyridyl. Aryl groups may be optionally substituted through available carbon atoms with 1 to about 4 groups. Exemplary substituents may include, but are not limited to, alkyl, halo, haloalkyl, alkoxyl, alkylthio, hydroxyl, methoxy, carboxyl, carboxylate, oxo, ether, ester, epoxy, alkyloxycarbonyl, alkylcarbonyloxy, amino, carbamoyl, urea, alkylurea, thioester, amide, nitro, carbonyl, and thiol. The aromatic groups may be heteroaryl. “Heteroaryl” refers to an optionally substituted aromatic ring system that includes at least one, and preferably from 1 to about 4 sulfur, oxygen, or nitrogen heteroatom ring members.

“Polyethylene glycol,” “PEG,” and “poly(ethylene glycol),” as used herein, refer to compounds of the structure “—(OCH₂CH₂)ₙ—” where (n) ranges from 2 to about 4000. The PEGs of the instant invention may have various terminal or “end capping” groups. The PEGs may be “branched” or “forked”, but are preferably “linear.”

The following examples are provided to illustrate various embodiments of the present invention. They are not intended to limit the invention in any way.

### Example 1

### Synthesis and Characterization of Alendronate Cyclodextrin

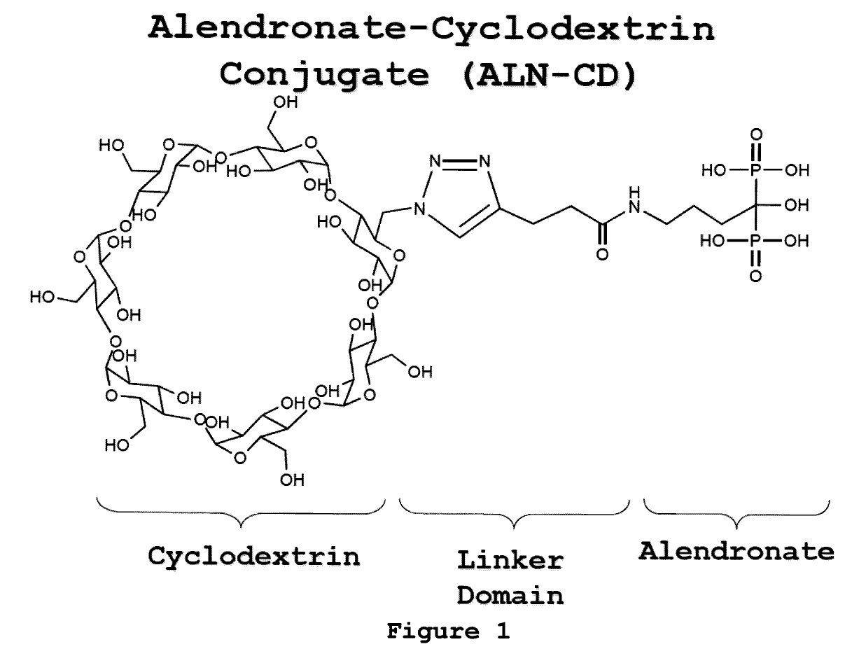

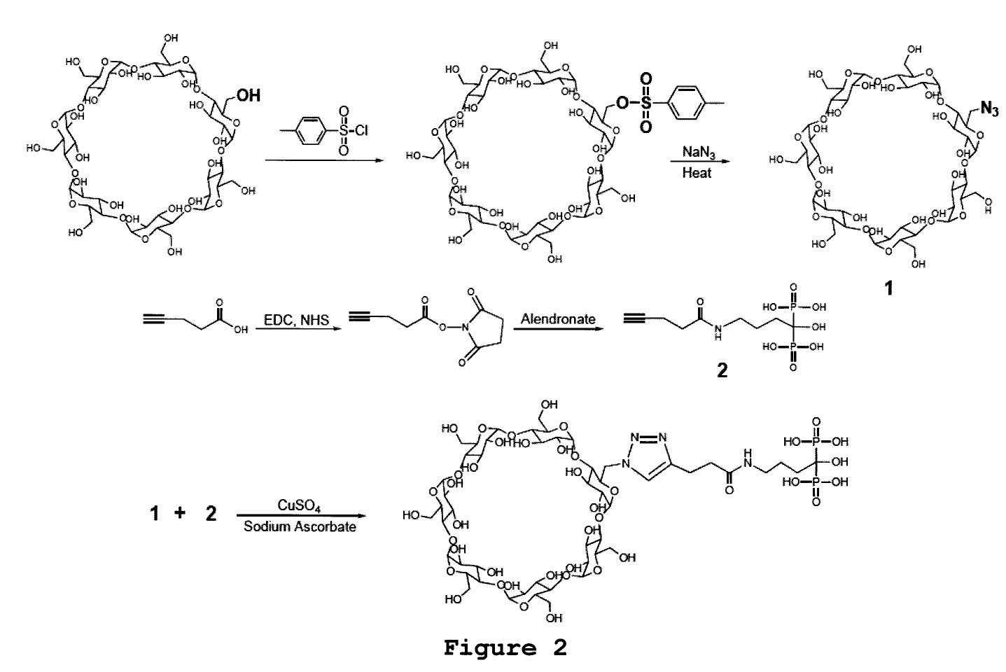

FIG. 1 is a schematic drawing of an alendronate cyclodextrin of the instant invention. FIG. 2 provides a schematic of the synthesis of alendronate cyclodextrin. This method of synthesis is described hereinbelow along with characterization studies of the resultant alendronate cyclodextrin.

### Reagents

Dexmethasone (Dex), prostaglandin E1, and β-cyclodextrin were purchased from TCI America (Portland, Oreg.). p-Toluenesulfonyl chloride, 4-pentynoic acid, 1-ethyl-3-(3-dimethylaminopropyl)carbodiimide hydrochloride (EDC), N-hydroxysuccinimide (NHS), sodium azide, CuSO₄.5H₂O, sodium ascorbic acid, dimethylformamide, and dichloromethane were purchased from Acros (Pittsburgh, Pa.). Alendronate was purchased from Ultratech India Ltd. (Vashi, New Mumbai, India). The internal standard, fluorometholone, was obtained from Sigma (St. Louis, Mo.). Ethanol and acetonitrile were obtained from Fisher (Pittsburgh, Pa.).

### Synthesis of Mono-6-(p-tolylsulfonyl)-β-cyclodextrin

β-cyclodextrin (120.0 g, 105.8 mmol) was suspended in 800 ml of water. NaOH (13.14 g, 328 mmol) in 40 ml water was added dropwise. The suspension became homogeneous before the addition was complete. p-Toluenesulfonyl chloride (20.16 g, 105.8 mmol) in 60 ml of acetonitrile was added dropwise. After 4 hours of reaction at room temperature the precipitate was removed by filtration and 8 mmol diluted HCl was added into the filtrate. The filtrate was then refrigerated overnight at 4° C. The resulting white precipitate was collected by filtration and dried, yielding the crude product. The pure product was obtained by recrystallization in hot water. Yield: 10%. ¹H NMR (500 Hz, DMSO-d₆) δ 7.75 (d, J=8.3 Hz, 2H), 7.43 (d, J=8.3 Hz, 2H), 5.83-5.63 (m, 14H), 4.85-4.77 (m, 7H); 4.52-4.17 (m, 6H), 3.70-3.42 (m, 28H), 3.39-3.20 (m, overlaps with HOD), 2.43 (s, 3H) ppm.

### Synthesis of Mono-6-(azido)-β-cyclodextrin (N₃—CD)

TsO-CD (6.44 g, 5 mmol) was suspended in water (50 ml) at 80° C., and sodium azide (3.25 g, 50 mmol) was added. The reaction was carried out with stirring at 80° C. for 6 hours. After being cooled to room temperature, the solution was poured into acetone (300 ml). The resulting precipitate was dried in vacuum to give the azide product as a white powder. The product was purified by dialysis (MWCO 500 dialysis tube). Yield: 80%. ¹H NMR (500 Hz, DMSO-d₆) δ 5.78-5.62 (m, 14H), 4.88-4.82 (m, 7H), 4.53-4.46 (m, 6H), 3.76-3.55 (m, 28H), 3.41-3.26 (m, overlaps with HOD) ppm.

### Synthesis of Active Ester (pentynoic acid 2,5-dioxo-pyrrolidin-1-yl ester)

2.0 g (20 mmol) of 4-pentynoic acid was dissolved in 80 ml CH₂Cl₂. 2.54 g (22 mmol) of N-hydroxysuccinimide (NHS) was added. Then, 1-ethyl-3-(3-dimethylaminopropyl)carbodiimide hydrochloride (EDC) was added (4.22 g, 22 mmol). The reaction was stirred at room temperature overnight. The reaction mixture was concentrated and the pure product was separated by silica gel column (hexane:ethyl acetate=2:1). Yield: 85%. ¹H NMR (500 Hz, CDCl₃) δ 2.88-2.83 (m, 6H), 2.60 (td, J₁=2.44 Hz, J₂=7.81 Hz, 2H), 2.04 (t, J=2.44 Hz, 1H) ppm.

### Synthesis of Conjugate of Alendronate and 4-Pentynoic acid (1-hydroxy-4-pent-4-ynamidobutane-1,1-diyldiphosphonic acid)

Alendronate (3.15 g, 10 mmol) was dissolved in 60 ml water (pH 7.0 or PBS), then 1.976 g (5 mmol) pentynoic acid 2,5-dioxo-pyrrolidin-1-yl ester in acetonitrile was added dropwise into this solution. The reaction was stirred at room temperature for 4 hours, then another 1.976 g (5 mmol) pentynoic acid 2,5-dioxo-pyrrolidin-1-yl ester in acetonitrile was added dropwise into this solution. After stirring at room temperature for 4 hours, 0.8 g (2 mmol) pentynoic acid 2,5-dioxo-pyrrolidin-1-yl ester in acetonitrile was added dropwise into this solution. The reaction was allowed to continue for 4 hours. The reaction solution was concentrated and precipitated in ethanol 3 times to give the final pure product. Yield: 90%. ¹H NMR (500 Hz, D₂O) δ 3.20 (t, J=6.84 Hz, 2H), 2.44 (m, 4H), 2.37 (t, J=2.44 Hz, 1H), 1.90 (m, 2H), 1.80 (m, 2H) ppm.

### Synthesis of Conjugate of Alendronate and Cyclodextrin (ALN-CD)

A 100 ml flask was charged with a magnetic stir bar, the aqueous 1-hydroxy-4-pent-4-ynamidobutane-1,1-diyldiphosphonic acid solution (1.38 g, 3.5 mmol), CuSO₄.5H₂O (125 mg, 0.5 mmol), and a freshly prepared aqueous solution of sodium ascorbic acid (0.99 g, 5 mmol). The mixture was allowed to stir at room temperature for 30 minutes. To this mixture was then added dropwise the mono-6-(azido)-β-cyclodextrin (N₃—CD) (4.64 g, 4 mmol) in H₂O. The reaction mixture was allowed to stir for 3 days at room temperature. The reaction solution was centrifuged at 4000 rpm for 0.5 hour and the supernatant was precipitated in DMF. After filtration, the supernatant was concentrated and precipitated in ethanol 3 times. Yield 82.5%. ¹H NMR (500 Hz, D₂O) δ 7.80 (s, 1H), 5.15-4.93 (m, 7H), 4.00-3.75 (m, 28H), 3.69-3.51 (m, 14H), 3.16 (t, J=6.79 Hz, 2H), 2.99 (t, J=7.32 Hz, 2H), 2.60 (t, J=7.32 Hz, 2H), 1.89 (m, 2H), 1.77 (m, 2H) ppm.

### Binding Potential of ALN-CD on HA

20 mg rhodamine B labeled ALN-CD or CD and 1 mg rhodamine B were dissolved in 0.5 ml water separately, and 100 mg of hydroxyapatite (HA) was added. The mixture was then allowed to stir gently for 10 minutes at room temperature. HA was recovered by centrifugation (10,000 rpm, 2 minutes), then washed with H₂O 5-10 times to remove unbound compounds. The HA was allowed to dry under vacuum at room temperature.

### Binding Rate of ALN-CD on HA

10 mg rhodamine B modified ALN-CD was dissolved in 25 ml water and the spectrum was recorded on UV-visible spectrophotometer. 20 mg HA was added into 1 ml of this solution and shaken for 0.5, 1, and 2 minutes. The solution was then centrifuged for 30 seconds and the supernatant was analyzed with UV.

### Phase Solubility of Dexamethasone or Prostaglandin E1 (PGE1) in the Presence of ALN-CD

Solubility studies were carried according to the method reported by Higuchi and Connors (Adv. Anal. Chem. Instrum. (1965) 4:117-212). Excess amounts of dexamethasone (3.92 mg) or PGE1 (2 mg) was added to aqueous solutions (1.0 ml) containing various concentrations of ALN-CD (from 0 to 10 mM). The experiments were carried out in triplicate. Tubes containing the solutions were sealed and shaken at 25° C. for 3 days. Suspensions were then filtered using a syringe through 0.22 μm filter. The concentration of dexamethasone or PGE1 in the filtrate was determined by HPLC equipped with a UV detector. For dexamethasone, 10 μg/ml fluorometholone was used as the internal standard.

The stability constant K was calculated with the following equation: K_{c}=slope/intercept×(1−slope), where slope is the slope of the phase solubility diagram and the intercept is the solubility of dexamethasone in water in the absence of ALN-CD.

The conditions for detecting dexamethasone were as follows: chromatographic column: Agilent C₁₈ reverse-phase (4.6×250 mm, 5 μm; Santa Clara, Calif.); mobile phase: acetonitrile-water (40:60, V/V) at a flow rate of 1 ml/min; UV detection at 240 nm.

The conditions for detecting PGE1 were as follows: chromatographic column: Agilent C₁₈ reverse-phase (4.6×250 mm, 5 μm); mobile phase: acetonitrile-0.01M KH₂PO₄ (42:58, v/v) at a flow rate of 1 ml/minute; UV detection at 205 nm.

### Preparation of Inclusion Complex

Inclusion complexes of the dexamethasone or PGE1 with ALN-CD were prepared at different molar ratios by mixing acetone or methanol solutions of dexamethasone or PGE1 with aqueous solutions ALN-CD of different concentrations. The resulting solutions were stirred at an ambient temperature until complete evaporation of the solvent. The suspensions were then filtered using a syringe through 0.22 μm filter, and the filtrate was lyophilized.

### Preparation of the Physical Mixtures

Physical mixtures were prepared in the same stoichiometric ratio as the complex obtained. Dexamethasone was mixed with ALN-CD in a mortar until a homogeneous mixture was obtained.

### Differential Scanning Calorimetry (DSC) of the Complex of PGE1 and ALN-CD

DSC of PGE1, ALN-CD and their complexes were performed in the temperature range of 30° C. to 180° C. using a Shimadzu DSC-50 Thermal Analyzer. The calorimeter was calibrated with various standards covering a range of temperatures exceeding those over which the studies were performed. Samples were sealed in an aluminum pan for analysis and an empty pan was used as a reference. Thermograms were recorded at a scanning speed of 5° C./minute under a nitrogen stream.

Characterization of the Dexamethasone Sodium Phosphate (DSP) Inclusion Complexes with ALN-CD by NMR

¹H NMR measurements were performed with a Bruker spectrometer (Billerica, Mass.). To prove the inclusion of dexamethasone in the ALN-CD cavity, DSP (15.5 mM) and ALN-CD (7.7 mM-46 mM) were dissolved in deuterated water. The internal reference was a peak due to small amounts of DHO and H₂O.

### Preliminary In Vitro Release Study

Dexamethasone (15 mg) or PGE1 (7.5 mg) and ALN-CD (100 mg) or CD (73 mg) complexes were studied in 4 ml H₂O solutions. The suspensions were filtered using 0.22 μm syringe filter and 500 mg HA was then added into the filtrates. The mixtures were vortexed for at least 10 minutes and then filtered and dried to give Dex or PGE1 loaded HA. 100 mg Dex or PGE1 loaded HA samples were extracted with 1 ml PBS (pH 7.4, 10 mM) for 10 minutes and analyzed by HPLC. Another 1 ml PBS was added to the Dex or PGE1 loaded HA and extracted 10 minutes for analysis.

The conditions for detecting dexamethasone were as follows: chromatographic column: Agilent C₁₈ reverse-phase (4.6×250 mm, 5 μm); mobile phase: acetonitrile-water (40:60, V/V) at a flow rate of 1 ml/min; UV detection at 240 nm.

The conditions for detecting PGE1 were as follows: chromatographic column: Agilent C₁₈ reverse-phase (46×250 mm, 5 μm); mobile phase: acetonitrile-0.01M KH₂PO₄ (42:58, v/v) at a flow rate of 1 ml/min; UV detection at 205 nm.

### Results

In the HA binding studies, the color of HA with rhodamine B and rhodamine B modified CD disappeared after ten studies. However, the color of with rhodamine B modified ALN-CD did not disappear with the washings, thereby indicating that ALN-CD successfully bound to the HA surface. Additionally, ALN-CD very quickly binds to the HA surface as evidenced by the almost complete saturation within 1 minute, as determined by the UV-visible spectra of rhodamine B labeled ALN-CD in the supernatant after incubation with HA.

The aqueous solubility of dexamethasone or PGE1 increases as a function of the concentration of ALN-CD. The solubility diagrams can be classified as A_{L} type according to Higuchi and Connors (Adv. Anal. Chem. Instrum. (1965) 4:117-212). Both diagrams are straight lines with a slope of less than 1, which may be ascribed to the formation of complexes in solution with 1:1 stoichiometry. The apparent 1:1 stability constant K_{c} calculated using the above equation rendered values of 2.57×10³ M⁻¹ and 4.78×10³ M⁻¹ for dexamethasone and PGE1 with ALN-CD, respectively. The determined 1:1 stoichiometry for both the complexes of ALN-CD with dexamethasone and PGE1 is similar to that previously reported for a complex of β-CD with dexamethasone (Shinoda et al. (1999) Drug Dev. Ind. Pharm., 25:1185-1192) and HP-β-CD with PGE1 (Gu et al. (2005) Int. J. Pharm., 290:101-108).

With regard to the DSC thermograms, PGE1 shows a characteristic endothermic fusion peak at approximately 116° C. The thermograms for ALN-CD exhibit a dehydration process that takes place about 80° C. The DSC thermograms for the physical mixtures ALN-CD and PGE1 show peaks corresponding to the pure ALN-CD and PGE1, thereby indicating the absence of an interaction between the compounds. In the case of the complex obtained by lyophilization, the endothermic peak around 116° C. disappears, indicating the inclusion of PGE1 in the cavity of ALN-CD.

NMR has shown the potential to provide almost complete information on guest-host interactions (stoichiometry, binding constants, energy of the complexation process, and structure of the complexes) in solution and in solid state (Chankvetadze et al. (1999) Ligand-cyclodextrin complexes. In: NMR Spectroscopy in Drug Development and Analysis. Weinheim, Germany: Wiley-VCH Verlag GmbH, pp 155-174). This information may be obtained mainly using ¹H NMR experiments based on the chemical shifts that show the protons of the drug and the CD when the inclusion occurs. Here, ¹H NMR was used to characterize the interaction in water of DSP with ALN-CD. Chemical shift changes of the protons of DSP in increasing concentrations (1:0 to 1:3 mol/mol DSP-ALN-CD) of the ALN-CD were analyzed.

The induced chemical shift changes for the hydrogen atoms of DSP whose signals were not masked by the ALN-CD signals as a function of the ALN-CD concentration were determined. A negative sign of Δ (ppm; i.e., the difference in DSP chemical shifts in the presence and absence of ALN-CD) indicates an upfield displacement and a positive sign indicates a downfield one. Downfield shifts of the protons of DSP are caused by variations of the local polarity due to the inclusion in the ALN-CD cavity (Echezarreta-Lopez et al. (2002) J. Pharm. Sci., 91:1536-47). C₂—H and C₁—H showed upfield shifts and C₄—H proton showed almost no chemical shift change, thereby indicating that these protons are near the edge of the annuli of the cyclodextrin. In contrast, C₁₁—H, C₂₁—H, C₇—H, C₁₄—H, C₁₅—H and methyl protons from carbons C₂₀—CH₃, C₁₈—CH₃, and C₁₉—CH₃ moved downfield, indicating their location inside the cyclodextrin cavity. These results suggest that in the complexes, the orientation of the protons is as follows: B, C, D ring protons are located inside the ALN-CD cavity. The A ring protons may interact with the edge of the ALN-CD and result in an upfield shift, but the A ring protons are not located inside the ALN-CD cavity because there is no chemical shift change for the C₄—H proton.

ALN-CD/PGE1 and ALN-CD/Dex complexes can bind strongly with HA through the bisphosphonate group. However, the controls CD/PGE1 and CD/Dex complexes would be predicted to only have non-specific binding with HA. Indeed, the in vitro release studies demonstrated that upon extraction, ALN-CD/PGE1 and ALN-CD/Dex complexes bound to HA release drug at a much slower rate than CD/PGE1 and CD/Dex complexes.

Therefore, CD can be chemically modified, such as by adding alendronate, without negatively impacting the hydrophobic cavity and its ability to complex with other compounds.

### Example 2

### In Vivo Studies with Alendronate Cyclodextrin

To determine the safety profile of the delivery system, a toxicity study was performed. Beta-cycldextrin (380 mg/kg), alendronate (100 mg/kg, LD50 dose) and ALN-CD (500 mg/kg) (molar ratio of 1:1:1) were all injected IV into BALB/c mice (3 per group, 20 g/mouse). All animals died within 7 days after administration except for the ALN-CD group which survived until the time of euthanasia without any noticeable side effects.

The effect of bone anabolic agent prostaglandin E₁ (PGE₁) in a cyclodextrin complex, with (PGE₁/ALN-CD) or without (PGE₁/hydroxypropyl(HP)-β-CD) a bone-targeting moiety (alendronate (ALN)), was evaluated on mandibular bone growth and inflammation. Specifically, a bilateral rat mandible model was used with test and control samples on contralateral sides. The test groups comprised: 1) one injection of PGE₁/ALN-CD (with 0.75 mg of PGE₁) vs. 2) PGE₁/HP-β-CD (with 0.63 mg of PGE₁) (n=6); 3) a graft of particulate hydroxyapatite (BioOss®, 20 mg) coated with PGE₁/ALN-CD (contains 138.11 μg PGE₁) vs. 4) BioOss® (20 mg) coated with PGE₁/HP-β-CD (contains 25.62 μg PGE₁) (n=6); 5) one injection of ALN-CD vs. 6) HP-β-CD (n=4); 7) one injection of PGE₁/ALN-CD (ALN-CD=20 mg; with 0.75 mg of PGE₁) vs. 8) ALN-CD (ALN-CD=20 mg) (n=6); 9) PGE₁ in EtOH (0.75 mg PGE₁) vs. 10) EtOH; 11) saline vs. 12) untreated; and 13) alendronate (ALN, 4.05 mg) vs. 14) saline. The rats were euthanized at 24 days and evaluated histomorphometrically at 24 days. Female Sprague Dawley rats, retired-breeder were used in these studies as they exhibit very little bone growth.

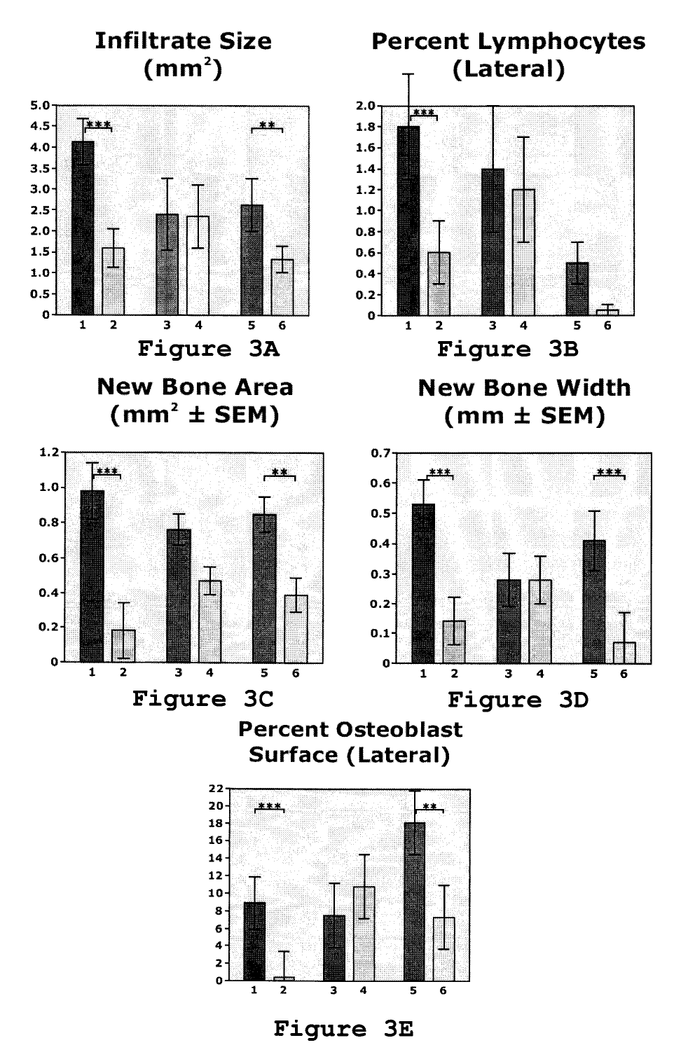

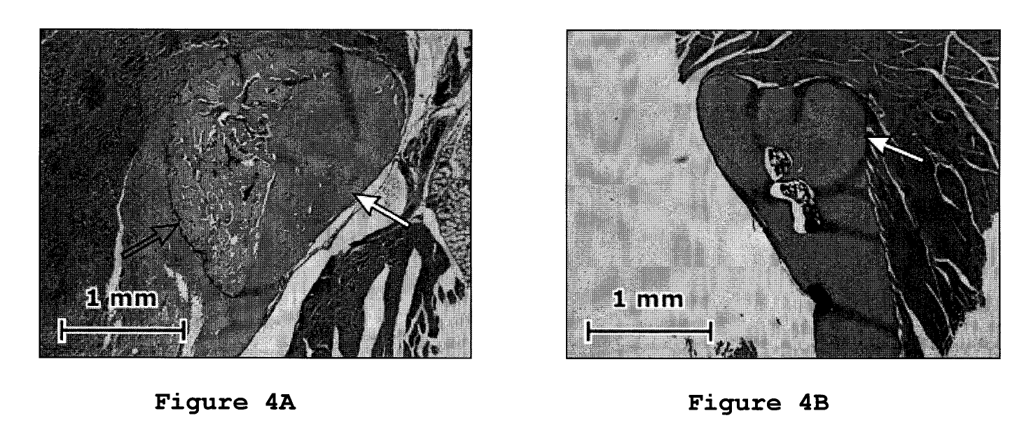

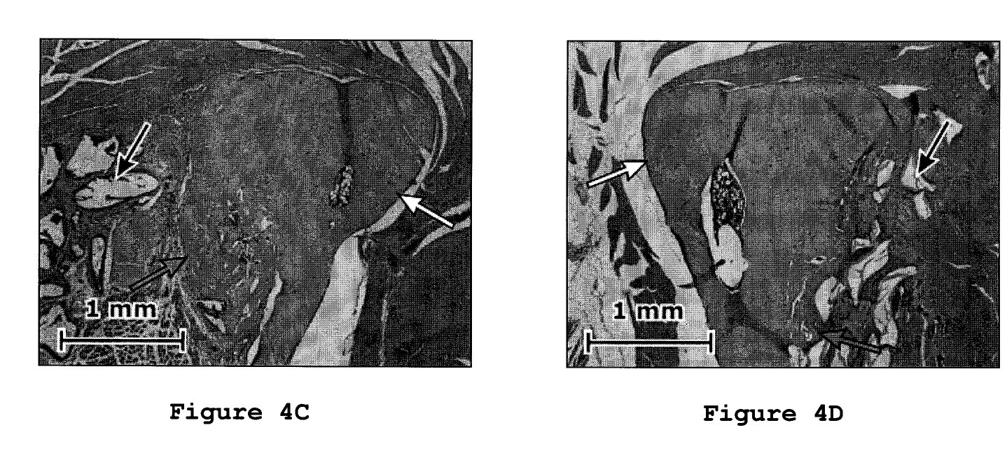

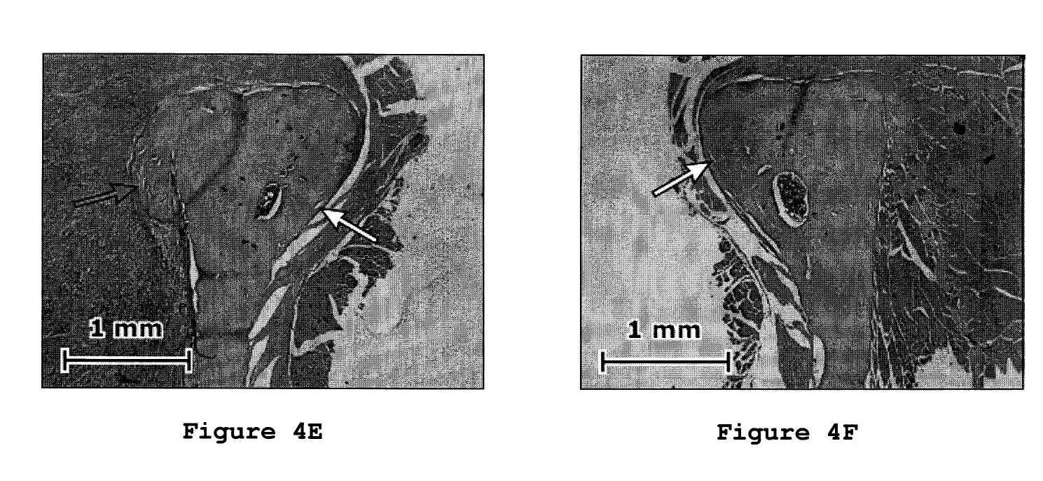

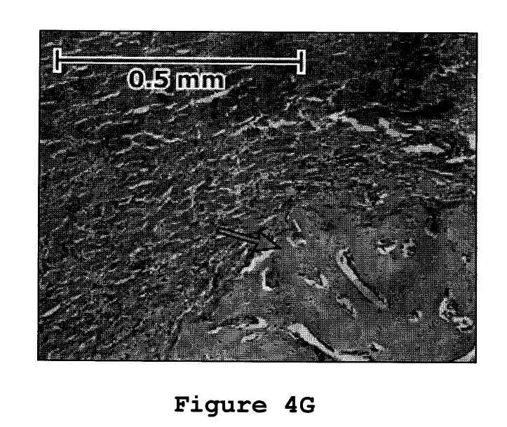

Injected PGE₁/ALN-CD vs. PGE₁/HP-β-CD sites had an increase in new bone width of 0.53±0.08 mm vs. 0.14±0.08 mm (p=0.0021), and an increase in the percentage of osteoblasts on the lateral periosteal surface of 8.9% vs. 0.4% (p=0.048) (Table 1 and FIG. 3). Surprisingly, ALN-CD vs. HP-β-CD sites also showed an increase in new bone width of 0.41±0.10 mm vs. 0.07±0.10 mm (p=0.024), and an increase in the percentage of osteoblasts of 18.1% vs. 7.3% (p=0.040). Injected PGE₁/ALN-CD had a larger area of inflammatory infiltrate than PGE₁/HP-β-CD (4.13±0.58 mm² vs. 1.60±0.58 mm², p=0.003), comprised of significantly increased percentages of neutrophils (up to 8.1%, p=0.04) and lymphocytes (up to 2.2%, p=0.0006). The groups where PGE₁/ALN-CD and PGE₁/HP-β-CD were absorbed in hydroxyapatite grafts (BioOss®) showed little bone growth and no difference between test and control sides overall, which was mainly due to the fact that the particles are not secured around the mandibular bone. However, when the grafts were secured around the mandibular bone, strong new bone growth was observed (FIGS. 4C and 4D).

To clarify the anabolic effect of ALN-CD found in 5) vs. 6), experimental groups 7) vs. 8); 9) vs. 10); 11) vs. 12); and 13) vs. 14) were performed. As shown in Table 1, it is very clear that ALN-CD itself could cause very robust new bone growth, which is even higher than its molecular complex with PGE₁. The new bone growth caused by direct PGE₁ injection is negligible. Injection of saline or EtOH could not cause any bone response, which ruled out the potential impact of mechanical stimulation (needle contact with bone surface) that may cause bone growth in other animal models.

Interestingly, alendronate injection caused moderate bone anabolic effect in the rat mandible model. A comparison between alendronate cyclodextrin conjugate (ALN-CD) and alendronate alone in saline (ALN) suggests (Table 1) that using formulation with equivalent amounts of ALN, ALN-CD caused more new bone area (1.11+0.25 mm²) than ALN (0.61+0.12 mm²). In addition, new bone width was greater in ALN-CD animals (0.47+0.14 mm) than ALN (0.14+0.05 mm) adjacent to where the formulations were injected (Table 1). Rats were injected with either a 50 μl of a 400 mg/mL solution of ALN-CD or 50 μl of an 81 mg/ml solution of ALN. Significantly, ALN-CD caused new bone to be deposited on the lateral surface of the mandible, which is the location of injection, in 6 of 6 cases. In contrast, ALN alone showed new bone in this area in only 5 of 8 cases. ALN also produced new bone on other distant areas of the mandible (e.g., the medial surface) in 8 of 8 cases. Significantly, ALN-CD did not cause bone formation in this area.

Taken together, these data indicate that the alendronate-cyclodextrin conjugate (ALN-CD) demonstrated a very strong and localized bone anabolic effect with a mechanism independent of the biological effect of alendronate and PGE₁. This characteristic allows for using injections of ALN-CD to repair isolated bone defects such as those found with periodontal disease and general bone fracture. It also holds the promise of treating systemic skeletal defects such as osteoporosis. Its tissue specificity in administration would reduce drug dose required and potential unwanted side effects.

Provided below is a summary of the bone formation in rat mandible in tabular form.

TABLE 1

| | | New Bone | New Bone |
|---|---|---|---|
| | New Bone Area | Width-1 | Width-3 |
| Groups | (mm² ± SEM) | (mm ± SEM) | (mm ± SEM) |
| ALN-CD/PGE₁ | 0.97 ± 0.23 | 0.50 ± 0.14 | 0.17 ± 0.06 |
| CD/PGE₁ | 0.18 ± 0.09 | 0.14 ± 0.06 | 0.16 ± 0.06 |
| P | 0.00001 | 0.00001 | NS |
| ALN-CD | 0.78 ± 0.10 | 0.36 ± 0.07 | 0.18 ± 0.03 |
| CD | 0.25 ± 0.08 | 0.05 ± 0.02 | 0.19 ± 0.11 |
| P | 0.003 | 0.0002 | NS |
| ALN-CD/PGE₁ | 0.66 ± 0.15 | 0.23 ± 0.05 | 0.26 ± 0.13 |
| ALN-CD | 1.11 ± 0.25 | 0.47 ± 0.14 | 0.37 ± 0.14 |
| P | 0.02 | 0.008 | NS |
| ALN | 0.61 ± 0.12 | 0.14 ± 0.05 | 0.24 ± 0.11 |
| Saline | 0.008 ± 0.008 | 0 | 0.02 ± 0.02 |
| P | 0.0004 | 0.06 | 0.005 |

### Example 3

### Multifunctional PEG

In contrast to other water-soluble biocompatible polymers, such as N-(2-hydroxypropyl)methacryl amide (HPMA) copolymer (Kopecek et al. (2000) Eur. J. Pharm. Biopharm., 50:61-81) and polyglutamic acid (PGA; Li, C. (2002) Adv. Drug Deliv. Rev., 54:695-713), the functionality of PEG is limited to its two chain termini regardless of the molecular weight. In order to overcome this limitation, approaches have been made to synthesize linear multifunctional PEGs (Nathan et al. (1994) Bioact. Compat. Polym., 9:239-251; Pechar et al. (2000) Bioconjugate Chem., 11:131-139; Cheng et al. (2003) Bioconjugate Chem., 14:1007-1017; Kumar et al. (2004) J. Am. Chem. Soc., 126:10640-10644). The methods that have been developed so far all involve multiple reaction steps. The yields and molecular weights of the resulting product are relatively low. Described herein is a novel and simple approach for the synthesis of a linear multifunctional PEG using the copper(I)-catalyzed Huisgen 1,3-dipolar cycloaddition, a “click” reaction.

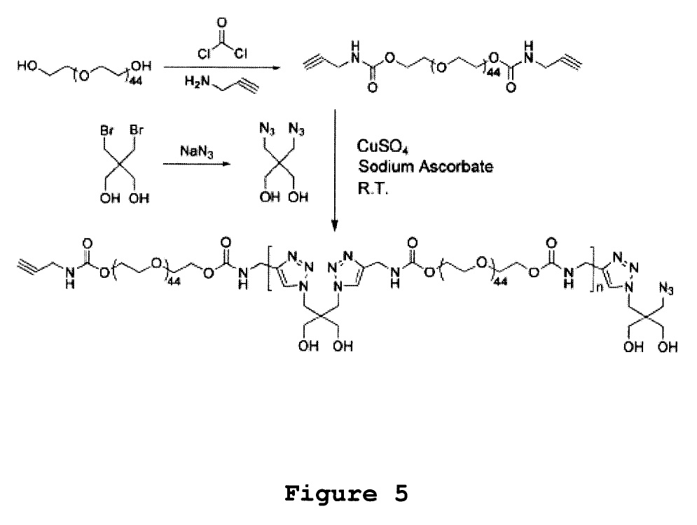

To achieve a simple and highly efficient synthesis of linear multifunctional PEG, a synthesis strategy was designed as shown in FIG. 5. PEG (MW=2000) diol is modified with propargyl amine. The acetylene-terminated PEG is then connected by 2,2-bis(azidomethyl)propane-1,3-diol with Cu(I) as the catalyst. Due to the self-catalyzing reaction that has been observed in “click” reactions using 2,2-bis(azidomethyl)propane-1,3-diol (Rodionov et al. (2005) Angew. Chem. Int. Ed., 44:2210-2215), this “click” polymerization is very efficient. The two hydroxyl groups of 2,2-bis(azidomethyl)-propane-1,3-diol will introduce pendent functionality to the resulting linear PEG. A more detailed chemical synthesis is provided in Example 4.

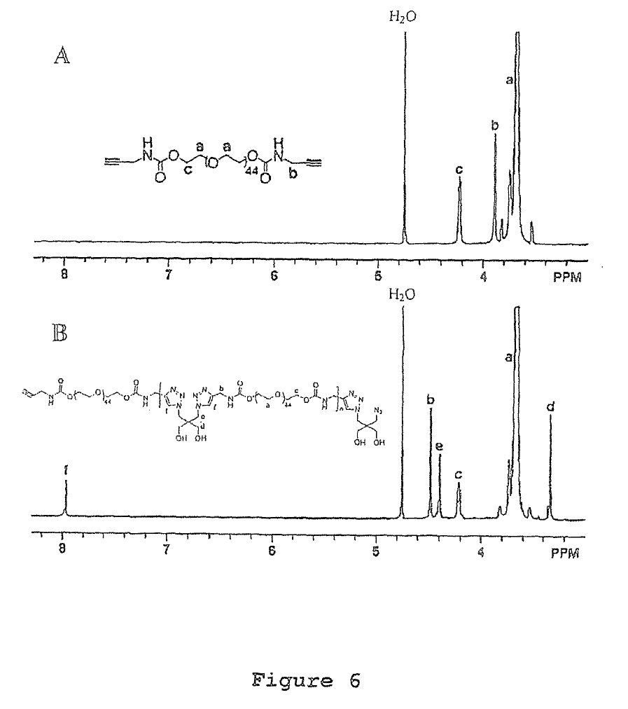

One critical step in preparation of linear, multifunctional PEG is to have 100% conversion of the two hydroxyl termini into acetylene (FIG. 5). PEG with mono-acetylene function will inevitably act as polymer chain terminator and lead to low molecular weight product. To activate the hydroxyl groups in PEG diol 2000, the dried PEG was first treated with phosgene (20% toluene solution). After removal of excess phosgene, propargyl amine was introduced. Acetylene-terminated PEG 2000 was then obtained via precipitation following the elimination of propargyl amine hydrochloride salt. To completely remove residual propargyl amine, the PEG product was further purified with LH-20 column. The structure of the modified PEG was confirmed by ¹H NMR analyses as shown in FIG. 6A.

The commercially available 2,2-bis-(bromomethyl)propane-1,3-diol may contain tribromide and tetrabromide. Therefore, triazide and tetraazide can be generated in the synthesis of 2,2-bis(azidomethyl)propane-1,3-diol. In the “click” polymerization, such tri- and tetra-functional linkers will lead to the formation of a cross-linked polymer network instead of a linear polymer. To avoid this, 2,2-bis-(bromomethyl)propane-1,3-diol was purified by repeated recrystallization in toluene and water. Its purity was confirmed by GC-MS. Azidation of 2,2-bis-(bromomethyl)-propane-1,3-diol was then carried out in DMF with sodium azide (FIG. 5).

The “click” polymerization of acetylene-terminated PEG 2000 (10 mM) with 2,2-bis(azidomethyl)propane-1,3-diol (10 mM) was performed in H₂O at room temperature as the reaction is particularly efficient in water (Rostovtsev et al. (2002) Angew. Chem. Int. Ed., 41:2596-2599; Bock et al. (2006) Eur. J. Org. Chem., 51-68). CUSO₄.5H₂O and sodium ascorbate (1.25 mM each) were used for in situ generation of the active Cu(I) as catalyst (Rodionov et al. (2005) Angew. Chem. Int. Ed., 44:2210-2215). The polymerization ended with gelation within 10 minutes. When the catalyst concentration was further reduced to 0.1 mM, gelation occurred overnight.

Without being bound by theory, two possible explanations for the observed gelation in the “click” polymerization are as follows. First, because the “click” reaction involves 2,2-bis(azidomethyl)propane-1,3-diol, which has a self-catalyzing effect (Rodionov et al. (2005) Angew. Chem. Int. Ed., 44:2210-2215), the polymerization could be highly efficient in forming high molecular weight PEG, thereby leading to gelation. Second, since triazole is a good electron donor, the newly formed triazole pair may interact with Cu(I) and form physical cross-links during the polymerization process. To explore the potential of the second possibility, the gel was washed extensively with EDTA solution (100 mM) with no gel dissolution observed over 24 hours. This rules out the possibility of a Cu(I) cross-linked polymer network. Therefore, the quick gelation observed in the “click” polymerization may be explained by the highly efficient reaction and the formation of very high molecular weight PEG.

To control the molecular weight and avoid gelation, propargyl amine (acetylene-terminated PEG:propargyl amine=9.5:1) was added into the reaction as a chain terminator (Odian, G. (2004) Principles of Polymerization 4th Ed, Wiley-Interscience, New Jersey, pp 74-80). A polymer solution was obtained under these conditions.

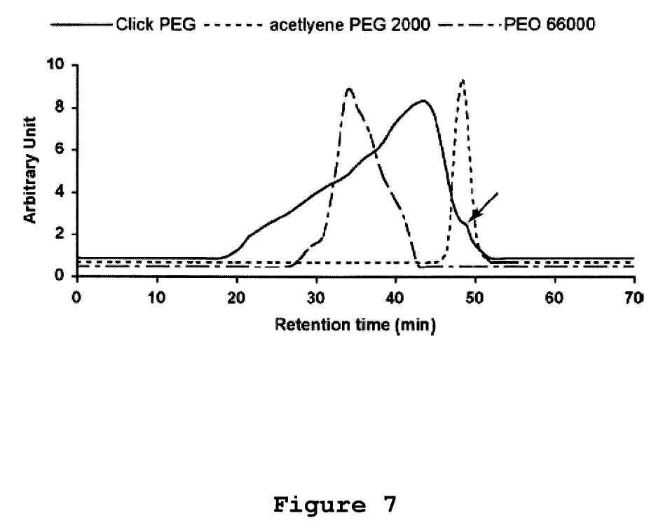

¹H NMR analysis of the polymer (after dialysis) shows the triazole proton at 7.97 ppm (peak f) and the methylene protons from the pendent diol structure at 3.34 ppm (peak d) and 4.39 ppm (peak e). In addition, the —CH₂— adjacent to the carbamate structure at 3.89 ppm [peak b (A)] shifts to 4.48 ppm [peak b (B)] after the “click” polymerization (FIG. 6). These clearly confirm the formation of linkages between each PEG 2000 segment. Size-exclusion chromatography (SEC) analysis (FIG. 7) of the product suggests that the resulting polymer (Click PEG) has high molecular weight and high polydispersity. Small amount of unreacted acetylene-terminated PEG 2000 is also evident in the SEC profile (FIG. 7, arrow).

In summary, a linear, multifunctional, high molecular weight PEG has been synthesized by Huisgen 1,3-dipolar cycloaddition from simple building blocks in water under very mild conditions. The reaction is simple and highly efficient. The molecular weight and polydispersity of the polymer can be controlled. Pendent diol groups have been successfully introduced to the linear PEG, which may be used directly to conjugate ketone (or aldehyde)-containing drugs to the polymer via pH-sensitive acetal structure. Since the “click” reaction has no interference with other functional groups, additional pendent structure such as —COOH and —NH₂ may also be introduced. Short segments of functional polymers (e.g. poly-N-isopropylacrylamide, polylysine or polyacrylic acid) may also be copolymerized with PEG to produce copolymers with unique biological and physicochemical properties. The instant “click” polymerization provides a unique opportunity to the development of novel polymers and functional polymer conjugates for a variety of biomedical applications.

### Example 4

### Chemical Synthesis of Multifunctional PEG

The following is an exemplary protocol for synthesizing multifunctional PEG of the instant invention.

### Materials

Polyethylene glycol (MW=2000) was purchased from Sigma (St. Louis, Mo.). 2,2-Bis-(bromomethyl)propane-1,3-diol and phosgene toluene solution (20%) were purchased from Aldrich (Milwaukee, Wis.). LH-20 resin and PD-10 columns were obtained from GE HealthCare (Piscataway, N.J.). Propargyl amine, sodium azide, sodium ascorbic acid, and copper sulfate were purchased from Acros (Moms Plains, N.J.). All solvents were purchased from Fisher Scientific (Pittsburgh, Pa.) or ACROS. ¹H NMR spectra were recorded on a 500 MHz NMR spectrometer (Varian, Palo Alto, Calif.). The weight average molecular weight (MW) and number average molecular weight (Mn) of copolymers were determined by size exclusion chromatography (SEC) using the AKTA™ FPLC system (GE HealthCare) equipped with UV and RI (Knauer; Berlin, Germany) detectors. SEC measurements were performed on Superose 6 columns (HR 10130) with PBS (pH=7.3) as the eluent.

Activation of Polyethylene Glycol (PEG) with Phosgene (COCl₂)

3 g of dried polyethylene glycol was dissolved in 10 ml of toluene in a round bottom flask (1.5 mmol). Phosgene was added in excess (12-15 ml of phosgene solution (20% in toluene); 5 mmol) to the flask with stirring. The reaction was allowed to proceed overnight in a closed fume hood. The excess phosgene was removed on a rotary evaporator.

### Synthesis of Acetylene Terminated Polyethylene Glycol

Propargyl amine (6 mmol, 0.33 g, 384.0 μL) was added to the reaction product of the above experiment after removal of excess phosgene. The reaction was allowed to proceed for 7-8 hours. The product was precipitated into diethyl ether. After precipitation, it was separated from the organic layer by centrifugation. The crude product yield is 95%. The product was further purified by dialysis (MWCO 2 k) and the product structure was confirmed by NMR and MALDI-TOF.

Alternatively, PEG diol 2000 (10 g, [—OH]=10 mmol) was dissolved in dry toluene, refluxed and dried in vacuum to remove water. Phosgene solution (15 ml, 20% in toluene) was then added into dried PEG with stirring. The reaction was allowed to precede overnight in a fume hood. The excess phosgene was removed in vacuum. DCM (20 ml) was used to dissolve the viscous residue. Propargyl amine (1.65 g, 30 mmol) was then added into the solution. The reaction was allowed to proceed for 7-8 hours at room temperature. The product was precipitated into diethyl ether 3 times and purified by LH-20 column. Yield: 83.3%. ¹H NMR (D₂O, 500 MHz): δ (ppm)=4.23 (t, PEG, —CH₂—), 3.89 (4 propargyl amide, —CH₂—), 3.68 (m, PEG, —CH₂—). To confirm the 100% derivatization of PEG diol into acetylene-terminated PEG, the product was also analyzed by ¹H NMR (CDCl₃, 500 MHz). No —OH signal (5=2.63 ppm) was detectable.

### Synthesis of 2,2-bis-(azidomethyl)-propane-1,3-diol

To a 50 ml round bottom flask was added 5 g of 2,2-bis-(bromomethyl)-propane-1,3-diol. 3 g of sodium azide was added to the flask with 10 ml of DMSO as the solvent for the reaction. The reaction was heated at 100° C. for 36 hours. The reaction was then cooled and water and brine was added. The mixture was extracted with ethyl acetate for five times and combined organic phases were washed with brine and dried over anhydrous magnesium sulfate. The final product was filtered and concentrated. The product obtained was a yellow oily liquid with 90% yield. Its structure was confirmed with NMR.

Alternatively, 2,2-bis-(bromomethyl)propane-1,3-diol (4 g, 15 mmol, recrystallized from toluene and water) was dissolved in DMF (30 ml). NaN₃ (4 g, 62 mmol) was then suspended in this solution. This mixture was stirred at 120° C. overnight and filtered to remove NaN₃ and NaBr. After the removal of DMF, dichloromethane (DCM, 20 ml) was added to the residue. The resulting precipitate was filtered off and the filtrate was evaporated to dryness. The residue was subjected to a standard diethyl ether/aq NaCl extraction. The organic phase was dried with Na₂SO₄ and evaporated to dryness. Then crude product was further purified by silica column (chloroform/methanol=20/1). Yield: 75.2%. ¹H NMR (CDCl₃, 500 MHz): δ (ppm)=3.61 (s, 4H), 3.41 (s, 4H), 2.75 (br, 2H).

### Click reaction between 2,2-bis-(azidomethyl)-propane-1,3-diol and acetylene terminated PEG

200 mg of PEG acetylene (0.092 mmol) was dissolved in a minimum amount of water (˜1.8 ml) in an ampoule. 20.0 mg (0.1 mmol) of 2,2-bis-(azidomethyl)-propane-1,3-diol was added to the above solution. 8 mg (0.06 mmol) of copper sulfate was subsequently added to the solution. 20 mg (0.10 mmol) of sodium ascorbate was added to the minimum amount of water and then this solution was added dropwise to the solution in the ampoule. In about 6 minutes, the polymerization solution become very viscous, indicating the formation of a high molecular weight polymer. To finish up the reaction, nitrogen was purged in the reaction vessel for a few minutes and then sealed. The reaction was allowed to proceed at 80-90° C. for 24 hours. FPLC was run to detect the high molecular weight multifunctional PEG, as comparing to the initial PEG (2 k).

Alternatively, acetylene-terminated PEG 2000 (205.2 mg, 95 μmol), 2,2-bis(azidomethyl)propane-1,3-diol (18.6 mg, 100 μmol), propargyl amine (0.55 mg, 10 μmol) and CuSO₄.5H₂O (3.13 mg, 12.5 μmol) were dissolved in H₂O (8 ml) with stirring. Sodium ascorbic acid (25 mg, 125 μmol) in H₂O (2 ml) was then added into this solution drop by drop. The reaction solution was stirred at room temperature for 4 hours. Before SEC analysis, the unreacted low molecular weight reactants were removed from the resulting polymer sample by PD-10 column. For large-scale purification and removal of unreacted PEG 2000, EDTA was added to the polymer solution and dialyzed against H₂O for 2 days. Molecular weight cutoff size of the dialysis tubing is 12 kDa of globular protein. After dialysis, the purified polymer product was lyophilized and analyzed by ¹H NMR. Yield: 66.9%. ¹H NMR (D₂O, 500 MHz): δ (ppm)=7.97 [s, triazole, —CH], 4.48 [s, triazole-CH₂-amide, —CH₂—], 4.39 [s, 2,2-bis(triazomethyl)propane-1,3-diol, —CH₂—], 4.21 [t, PEG, —CH₂—], 3.68 [m, PEG, —CH₂—], 3.34 [s, 2,2-bis(triazomethyl)propane-1,3-diol, —CH₂—].

In yet another alternative, the modified PEG may be generated without the chain terminator propargyl amine. Acetylene-terminated PEG 2000 (21.6 mg, 10 μmol), 2,2-bis(azidomethyl)propane-1,3-diol (1.9 mg, 10 μmol) and CuSO₄.5H₂O (0.31 mg, 1.25 μmol) was dissolved in H₂O (0.8 ml) with stirring. Sodium ascorbic acid (2.5 mg, 12.5 μmol) in H₂O (0.2 ml) was then added into this solution drop by drop. Gelation happens within 1 hour.

### Synthesis of Multifunctional Copolymer-Drug Conjugate

Dexamethasone may be reacted with the multifunctional copolymer in the presence of a crystal of toluene-p-sulfonic acid or trimethylsilyl chloride in methanol at room temperature (Chan et al. (1983) Synthesis 3:203-205). This will result in acetal bond formation at position 19.

As a secondary approach, dex may be first conjugated with 2,2-bis-(azidomethyl)-propane-1,3-diol. The resulting diazide may then be reacted with acetylene modified PEG to form the copolymer-DEX conjugate. The average molecular weight of polymeric conjugates may be determined by size exclusion chromatography (SEC) using the AKTA™ FPLC system (GE Healthcare) equipped with UV and RI (Knauer) detectors. SEC measurements may be carried out on Superdex 75 or Superose 6 columns (HR 10/30) with PBS (pH=7.3) as the eluent. The average molecular weights of the conjugates may be calculated using PEG homopolymer standards calibration.

### Biological Evaluation

After purification of the conjugate with LH-20 column fractionation (×2) to remove any free Dex from the conjugate, it can be incubated at 4, 25 and 37° C. in isotonic buffer systems of pH 5.0, 6.0 and 7.4 over a two weeks period of time. The release of free Dex can be monitored with an Agilent HPLC system (Diode array UV/Vis detector, 240 nm; Agilent C18 column, 4.6×150 mm, 5 pm; mobile phase: acetonitrile/water=50%/50%; flow rate: 0.5 ml/minute; injection volume: 10 μl) using a validated protocol.

A rat model can be used to compare the efficacy of Dex conjugate compared to free Dex (Wang et al. (2004) Pharm. Res., 21:1741-1749). Different PEG-Dex conjugates can be tested for optimal treatment conditions. In the treatment study, the volume of the arthritic joint and inflammation indices can be measured. The endpoints of bone mineral density, bone erosion surface and histopathological analysis can also be performed. These results can be compared with controls treated with free Dex and vehicle to demonstrate the full therapeutic potential of the delivery system.

Free Dex and Dex-PEG copolymer conjugates can be given to healthy male Lewis rats at different dosing schedules. At the end of the experiment, body weight, size, bone formation rates, mineral density and other bone histomorphological parameters of the skeleton can be analyzed for indications of side effects. Other soft tissues (adrenal gland, spleen, thymus, liver) can be isolated, weighed and analyzed histologically. These results can be compared with those from the control group treated with vehicle to demonstrate the superior safety profile of the novel delivery system.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. A compound of the formula T-X-CD, wherein X is a linker domain, T is bone targeting moiety, and CD is a cyclodextrin.

2. The compound of claim 1, wherein said bone targeting moiety is selected from the group consisting of a bisphosphonate, alendronate, tetracycline, sialic acid, malonic acid, N,N-dicarboxymethylamine, 4-aminosalicyclic acid, 4-aminosalicyclic acid, antibodies, antibody fragments, and peptides comprising about 2 to about 100 residues selected from the group consisting of D-glutamic acid, L-glutamic acid, D-aspartic acid, and L-aspartic acid.

3. The compound of claim 2, wherein said bone targeting moiety is alendronate.

4. The compound of claim 1, wherein said cyclodextrin is selected from the group consisting of α-CD, β-CD, γ-CD, μ-CD, dimethyl-β-CD, carboxymethyl-ethyl-β-CD, sulfobutyl-ethyl-β-CD, and hydroxypropyl-β-cyclodextrin.

5. The compound of claim 4, wherein said cyclodextrin is hydroxypropyl-β-cyclodextrin.

6. The compound of claim 1, wherein each cyclodextrin is linked to more than one bone targeting moiety.

7. The compound of claim 1, wherein said linker domain is selected from the group consisting of a bond, alkyl group, alkenyl group, aryl group, and polypeptide.

8. A composition comprising the compound of claim 1 and at least one pharmaceutically acceptable carrier.

9. The composition of claim 8, further comprising at least one bone related therapeutic agent.

10. The composition of claim 9, wherein said at least one bone related therapeutic agent is complexed within the hydrophobic cavity of the cyclodextrin of said compound.

11. A method of preventing or treating bone disorders and bone disorder-related conditions or complications in a subject in need thereof comprising administering to the patient the composition of claim 8.

12. The method of claim 11, wherein said bone disorder is selected from the group consisting of osteoporosis, osteopenia, bone fractures, bone breaks, Paget's disease (osteitis deformans), bone degradation, bone weakening, skeletal distortion, low bone mineral density, scoliosis, osteomalacia, osteomyelitis, osteogenesis imperfecta, osteopetrosis, enchondromatosis, osteochondromatosis, achondroplasia, alveolar bone defects, vertebra compression, bone loss after spinal cord injury, avascular necrosis, fibrous dysplasia, periodontal disease, hyperparathyroidism (osteitis fibrosa cystica), hypophosphatasia, fibrodysplasia ossificans progressive, and pain and inflammation of the bone.

13. The method of claim 11, wherein said composition is administered systemically.

14. The method of claim 11, wherein said composition is administered locally.

15. The method of claim 11, wherein said composition is administered by injection.

16. A method for synthesizing a multifunctional poly(ethylene glycol) (PEG) comprising:
a) providing a PEG wherein the termini of said PEG comprise a first functional group capable of participating in a click chemistry reaction;
b) contacting said PEG of step a) with a compound comprising a complementary second functional group capable of participating in a click chemistry reaction with said first functional group, under conditions which allow for the click chemistry reaction; and
c) isolating the resultant multifunctional PEG.

17. The method of claim 16, wherein the click chemistry reaction is a cycloaddition reaction.

18. The method of claim 17, wherein the cycloaddition reaction is a 1,3-dipolar cycloaddition reaction.

19. The method of claim 16, wherein said first functional group is an azide and said second functional group is an alkyne, or wherein said first functional group is an alkyne and said second functional group is an azide.

20. The method of claim 16, wherein said compound of step b) is 2,2-bis-(azidomethyl)-propane-1,3-diol and said first functional group is acetylene.

21. The multifunctional PEG generated by the method of claim 16.

22. The multifunctional PEG of claim 21 which is formula (I).

23. The multifunctional PEG of claim 21 conjugated to at least one therapeutic compound.

24. A composition comprising the multifunctional PEG of claim 21 and at least one pharmaceutical carrier.

25. The composition of claim 24 further comprising at least one therapeutic agent.

26. A method of treating arthritis comprising administering the composition of claim 24.
